(19) United States
(12) Patent Application Publication
(10) Pub. No.: US 2007/0299148 A1
(43) Pub. Date: Dec. 27, 2007

(54) Tubular Reactor With Packing

(76) Inventor: Guy Lode Magda Maria Verbist, Amsterdam (NL)

Correspondence Address: SHELL OIL COMPANY, P O BOX 2463, HOUSTON, TX, 772522463, US

(21) Appl. No.: 11/667,501

(22) PCT Filed: Nov. 11, 2005
(86) PCT No.: PCT/EP05/55896

§ 371 Date: May 10, 2007

(30) Foreign Application Priority Data
Nov. 12, 2004 (EP) 04105740.7

(51) Int. Cl. C07C 27/00

(52) U.S. Cl. 518/712; 518/719

(57) ABSTRACT

A multitubular fixed bed reactor suitable for carrying out a catalytic process is described. There actor includes a plurality of reactor tubes, one or more of which include a fixed bed of catalyst and is/are at least partially surrounded by a heat transfer medium, preferably a cooling medium, wherein the one or more reactor tubes include at least one insert, especially a heat transfer insert and/or a pressure drop decreasing insert. Preferably the tubes are elongate, and preferably the inserts are elongate. The inserts may not extend the full length of their respective tube or tubes. The inserts may extend partly, substantially or wholly along the length of the relevant tube(s). All the tubes of the reactor may include a heat transfer and insert. Most preferably, the insert of the present invention reduces temperature differentials in a reactor tube and decrease the pressure drop over the reactor tube, so as to minimise even temperature differences, and thus optimise reaction conditions and catalytic activity within the reactor tube.

The present invention relates to a multitubular fixed bed reactor suitable for carrying out catalytic processes, particularly, but not exclusively exothermic reactions such as the Fischer-Tropsch process.

The Fischer-Tropsch process can be used for the conversion of hydrocarbonaceous feed stocks into normally liquid and/or solid hydrocarbons (0° C., 1 bar). The feed stock (e.g. natural gas, associated gas, coal-bed methane, residual oil fractions, biomass and/or coal) is converted in a first step into a mixture of hydrogen and carbon monoxide (this mixture is often referred to as synthesis gas or syngas). The synthesis gas is then fed into a reactor where it is converted in a single step over a suitable catalyst at elevated temperature and pressure into paraffinic compounds ranging from methane to high molecular weight modules comprising up to 200 carbon atoms, or, under particular circumstances, even more.

Numerous types of reactor systems have been developed for carrying out the Fischer-Tropsch reaction. For example, Fischer-Tropsch reactor systems include fixed bed reactors, especially multi-tubular fixed bed reactors, fluidised bed reactors, such as entrained fluidised bed reactors and fixed fluidised bed reactors, and slurry bed reactors such as three-phase slurry bubble columns and ebulated bed reactors.

The Fischer-Tropsch reaction is very exothermic and temperature sensitive with the result that careful temperature control is required to maintain optimum operation conditions and desired hydrocarbon product selectivity. Indeed, close temperature control and operation throughout the reactor are major objectives.

The heat transfer characteristics of a fixed-bed reactor, i.e. a reactor filled with one or more packed beds of loose catalyst particles, are limited because of the relatively low mass velocity, small particle size and low thermal capacity of the fluids. If one attempts, however, to improve the heat transfer by increasing the gas velocity, a higher CO conversion could be obtained, but an excessive pressure drop across the reactor may develop, which limits commercial viability. In order to obtain the CO conversions desired and gas through-puts of commercial interest, the conditions result in substantial radial temperature gradients. For that reason, Fischer-Tropsch fixed-bed reactor tubes generally have had a diameter of less than 10 cm, or even less than 7 cm, to avoid excessive radial temperature differences.

In addition to the heat transfer characteristics of a fixed bed reactor, another problem is the pressure drop over the reactor tubes in a fixed bed reactor. Especially when the catalyst particles are small and/or the reactor bed is relatively long, pressure drops over the reactor tubes may be in the range of between 2 and 20 bar, more often between 5 and 10 bar. In reaction in which diffusion limitation is important, e.g. the Fischer-Tropsch reaction, there is a clear preference for small catalyst particles. The use of small particles improves the C₅₊-selectivity, but also results in a high pressure drop. Thus, another aspect of the invention is to reduce the pressure drop over the reactor. A reduced pressure drop requires less investment (a smaller compressor can be used and the pressure requirements of the equipment before the catalyst beds may be less severe) and will reduce operational costs (less energy is required).

The desired use of high-activity catalysts in Fischer-Tropsch fixed-bed reactors makes the situation even more challenging. The limited heat transfer performance makes local runaways (hotspots) possible, which may result in local deactivation of the catalyst. In order to avoid runaway reactions the maximum temperature within the reactor must be limited. However, the presence of temperature gradients within the reactor means that some of the catalyst may be operating at sub-optimal conditions. In addition to the radial temperature difference, there is usually also an axial temperature profile, resulting in an even more serious problem as to sub-optimal use of the catalyst. The use of high-activity catalyst also requires relatively small catalyst particles in order to obtain the desired C₅₊-selectivity. Such particles may result in very high pressure drops over the catalyst bed. These high pressure drops require large (expensive) compressors, and add a substantial power requirement to the operation of the plant.

The use of liquid recycles as a means of improving the overall performance in a fixed-bed design has been described. Such a system is also called a “trickle bed” reactor (as part of a sub-set of fixed-bed reactor systems) in which both reactant gas and liquid are introduced (preferably in an up flow or down flow orientation with respect to the catalyst) simultaneously. The presence of the flowing reactant gas and the liquid improves heat removal and temperature control, thus enhancing the reactor performance with respect to CO conversion and product selectivity. However, the liquid recycle also increase the pressure drop over the catalyst bed.

A potential limitation of the trickle bed system (as well as of any fixed-bed design) is the pressure drop associated with operating at high mass velocities. The gas-filled voidage in fixed-beds (typically less than 0.50) and size and shape of the catalyst particles do not permit high mass velocities without excessive pressure drops. Consequently, the conversion rate per unit reactor volume is limited by heat removal and pressure drop. Increasing the individual catalyst particle size may slightly improve the heat transfer rates by allowing higher mass velocities (for a given pressure drop), but the loss in selectivity towards the high boiling point products and the increase in methane selectively combined with the increase in catalyst activity generally offset the commercial incentives of higher heat transfer.

For some catalytic reactions it has been proposed to incorporate pieces of metal (relative small metal particles e.g. metal scraping or metal curls of a size comparable with the catalyst particles) or other heat conductive material in mixture with the catalyst in the catalyst bed to facilitate heat transfer. The reactants may also be diluted with non-reactive gases or vapours as a further means of achieving temperature control. The temperature can also be controlled by using low flow rates or low conversion levels so that the amount of heat generated is low, but this causes the yield per unit time to be low and the process therefore to be more expensive. Another possible way is to use (metal) inserts coated with catalyst particles. This results in a low catalyst loading and thus low productivity per volume unit. Increasing the particle size will (at a given space velocity) reduce the pressure drop, but due to mass transfer limitations inside the catalyst particle this may result in a lower production rate or a in a (less valuable) reaction product.

It is one object that the present invention to seek suppression of temperature profiles in fixed bed multitubular reactors and to reduce the pressure drop over the fixed bed reactors.

Accordingly, the present invention provides a multitubular fixed bed reactor suitable for carrying out a catalytic process, which reactor includes a plurality of reactor tubes, one or more of which include a fixed bed of catalyst and is/are at least partially surrounded by a heat transfer medium, preferably a cooling medium, wherein the one or more reactor tubes each include at least one insert. The insert functions as a heat transfer insert. The insert also functions as a pressure drop decreasing insert. The use of these inserts also reduces the pressure drop over the fixed bed catalyst.

The insert(s) can provide heat transfer both directly, by their conductivity, and indirectly, by their possible ability to influence the direction of flow of one or more of the reactants through the reactor tube. In addition, the use also results in a decrease of the pressure drop.

One or more tubes of the reactor of the present invention may include a plurality of inserts, which may or may not be the same or different. Any reference herein to an “insert” applies also to “inserts” and vice versa, whether such inserts are located in the same or different reactor tubes. Any reference herein to an “insert” may not apply equally to any plurality of “inserts”. One reactor tube may include different inserts, and different tubes may include different inserts.

The reactor tubes comprise a fixed bed of catalyst. The catalyst is suitably a particulate catalyst. The shape of the catalyst may be regular or irregular. The dimensions are suitably 0.5-30 mm in all three directions, preferably 2-20 mm in all three directions. Suitable shapes are spheres and, in particular, extrudates. The extrudates suitably have a length between 2 and 30 mm, preferably between 4 and 20. The cross section may be a circle or, preferably, a trilobe. The circle or the circle around the trilobe has suitably a diameter between 0.5 and 10 mm, preferably between 1 and 5 mm. The reactor tube is suitably filled by dumping the catalyst into the tube. In general, the reactor tube is completely filled over the full length of the tube, with the exception of the first 1 to 50 cm. On top of the catalyst bed a layer of larger, inert particles may be present. In principle, the distribution of the catalyst is homogeneous over the axial and the radial distribution of the catalyst in the reactor tube. The catalyst is especially a supported catalyst, the support preferably being a porous refractory oxide. Preferred refractory oxides are silica, alumina, titania and mixtures thereof. Porous refractory oxide supports are very suitable for carrying a highly dispersed metal catalyst, which is a highly active catalyst.

Preferably the tubes are elongate, and preferably the inserts are elongate. The inserts may not extend the full length of their respective tube or tubes. The inserts may extend partly, substantially or wholly along the length of the relevant tubes, or even beyond the length of the tube. All the tubes of the reactor may include a heat transfer insert. In a preferred embodiment the inserts extend to 90% of the top part of the catalyst bed, preferably 70%, more preferably 50%, as most of the heat is generated in the top section of the catalyst bed. In the case that often reduction of the pressure drop is the target, the length is preferably at least 50% of the length of the catalyst bed, preferably at least 75%, more preferably at least 90%. Shorter inserts are easier to install.

The inserts may have any suitable shape, design or pattern.

In a preferred embodiment the inserts have a straight or linear elongated shape, i.e. the insert does not show any helical or twisted deformations. In that way gas and liquid flows are not disturbed, and the conditions are more or less equal for all catalyst particles at a certain height in the reactor tube. For instance, the use of a helically wound insert would result in transportation of gas and liquid to the outside part of the tube. Thus, the liquid flow in the center of the reactor tube will be minimal, while the outside catalyst layer will be drowned in the liquid. Preferably the distance between the outer parts of the inserts and the reactor tube is very small, more preferably the outside part of the insert is in direct contact with the reactor tube. It is observed that the contact is preferably a “touching contact”, i.e. both parts touch to each other, and is not a “fixed contact”, e.g. as is the case of a soldered or a welded connection.

Preferably the inserts have a simple geometric cross-section, being of a structure which extends along the space of the reactor tube, and which has connectivity to provide heat transfer either radially across the tube or axially along the tube or both. The inserts may be regular or irregular in shape, design and/or pattern, and have a repeating or non-repeating shape, design and/or pattern. In one embodiment the inserts are symmetrical, although non-regular shapes and designs are also possible, such as foams, for example ceramic, sintered and metal foams.

In one embodiment of the present invention, the insert can have a central axis, and possibly comprise or have one or more radial arms, fins, or other projections extending therefrom. Examples include a star cross-sectional shape, having two or more radial fins, preferably, three, four or more radial fins. One or more of the radial fins, etc may or may not be of the same length, or extend radially outwardly, to the same extent as others, or have the same shape or design as other fins. Another example includes a ‘pipe cleaner’ shape, having a central axis and a number of fine radial projections, possibly intermittently, along its length.

Such projections may extend partly, substantially or fully along the length of the insert. The projections could extend angularly.

The insert may comprise one or more parts, portions or segments having a different cross-sectional shape or design than other parts, portions or segments.

The insert may also be linear, or may be helically or twisted etc, possibly having one or more strands or turns, along its length. In a preferred embodiment the insert are linear (or straight), i.e. they are not helically and/or twisted, thus resulting in a homogeneous distribution of the gas and liquid phase over the solid phase. The insert may also be a hollow or partly hollow shape, such as a cylinder or tubular polygon.

The insert may be perforated, for example by one or more openings, apertures and the like, in the insert, either regularly or irregularly along its length.

The insert may be partly, substantially or wholly porous. In this way, the insert is able to absorb and/or adsorb some of the material in the reactor tube, which absorption or adsorption increases the insert-boundary layer. This reduces the heat transfer coefficient at the boundary or interface of the insert, and so increases the heat transfer rate between the insert and the reactor tube content, making the insert more efficient in its heat transfer action.

One or more parts of the insert may have a greater or lesser thickness or diameter than other parts.

The parts, portions or segments of the insert may be wholly or substantially straight, or arcuate, or a combination thereof. One or more of the parts may also taper inwardly, outwardly, or both, along the insert.

The insert may include one or more radial parts or portions, having a substantial cross-sectional area compared with the overall cross-sectional area of the insert.

The insert may be made of any suitable material adapted to physically and chemically withstand the catalytic process, including but not limited to metals, preferably copper, iron, steel, aluminium and titanium, their alloys, plated materials such as copper-plated nickel, and ceramic alloys, as well as other materials used to form reactor vessels and tubes.

The insert may or may not be involved with the process, or include a coating or finishing, optionally including a material involved with the process being carried out in the reactor, such as a catalytically active material having a catalytic activity as herein described. Preferably the insert is made from an inert material, thus not further increasing the reaction.

The or each reactor tube may have a flow path for the general direction of the or each reactant passing therethrough, and/or the or each product formed by the reactant(s), as well as any other fluid(s) passing therethrough. The flow path may be the general direction of the or each tube. Preferably the reactor tubes have the same internal and external diameter over the full length. More preferably, all reactor tubes in a multitubular fixed bed reactor are identical.

The inserts may also be adapted to assist or influence the flow paths, of the or each reactant in the tube. This may be radially, axially or a combination. This includes the action of twisted blade mixers currently used. Such influence may also assist heat transfer within the tube. Straight (or linear) inserts are preferred. The inserts are preferably elongated inserts aligned with the reactor wall. In general, there are no protruding surfaces which act as flow obstructing parts. In that way the gas/liquid flow is not disturbed and no additional pressure drop is created.

The heat transfer medium could be for cooling or heating. It is generally a cooling medium, which could be water, steam, a combination thereof, or oil or molten salts. All the tubes in the reactor could be partially or wholly surrounded by such a medium.

The reactor tubes can be connected, or have a common feed(s) and/or outlet(s). The reactor may include one or more reactor sheets, heads or plates, perforated to accept the ends of the tubes.

The reactor of the present invention is typically useful for carrying out raised temperature catalytic processes, and exothermic reactions including the Fischer-Tropsch process and olefin oxidation, e.g. the oxidation of ethane or propane to epoxides.

The inserts may vary, influence or otherwise adapt the temperature directionally within the tube, in such a way as to provide a different temperature distribution within the tube than would occur without the insert. In addition, the pressure drop over the bed will be reduced.

The inserts may be adapted to transfer heat inwardly towards or outwardly from the direction of the centre of the tube in which the insert is located, that is generally substantially radially.

The heat transfer direction can also be axial. Some reactions in multitubular reactor tubes also have an axial temperature profile. For instance, the conversion of reactants may mean greater heat being generated towards the tops of the reactor tubes.

The insert(s) of the present invention can influence the axial temperature distribution or profile, either independently or in association with any radially temperature influence.

The ability of the present invention to provide one or more inserts, each individually or comparatively having variable shapes, designs and/or patterns, with or without such inserts also being able to provide one or more radially and/or axial divisions of the reactor tube, also allows the present invention to influence the catalytic activity radially across and/or axially along the tube. For example, the width of one insert or the width of different inserts may vary, such that the space for the catalyst varies axially. In another example, an insert(s) in the reactor tube may allow one or more areas, e.g. pockets, of non-catalytic activity, possibly radially across the tube.

The present invention further provides a tube suitable for use in a multitubular reactor for carrying out a catalytic process, which tube includes at least one heat transfer insert as hereinbefore defined.

The insert may be separate and/or removable from the tube. The insert may also extend beyond an end or ends of the tube, possibly in combination with an end cap or plate used to provide a divider or wall for the catalyst area, for example tube clips.

Preferably, the insert is locatable within a reactor tube following construction of the tube and/or reactor.

Also preferably, the insert is retrofitable within a reactor tube. Thus, the insert can be applied or located retrospectively to existing multitubular reactors without any redesign of the reactor or its tubes.

The present invention further provides a process for the synthesis of hydrocarbons comprising the step of introducing reactants into a multitubular reactor having a plurality of tubes in which at least one reactant is in contact with a catalyst, and is/are surrounded by a heat transfer medium, wherein at least one of the reactor tubes includes at least one heat transfer insert.

The process could be an endothermic or exothermic reaction, including the Fischer-Tropsch process. Preferably in a process such as Fischer-Tropsch, where the difference between the maximum temperature and minimum temperature across a horizontal cross-section of a catalytically active part of the tube can be between 10 to 30° C., using an insert of the present invention, compared with not using an insert, can provide a temperature difference of less than 50%, preferably less than 35%, more preferably less than 25%, for the same tube parameters.

This temperature difference consideration discounts the usually relatively large temperature variation in the boundary layer between the actual reactor tube wall, especially where the reactor tube is cooled or heated by an external heat transfer action, and the internal reactor tube reaction zone. In the present invention, this boundary layer next to the reactor tube wall is termed herein the mantle.

For example, in a Fischer-Tropsch reaction where the reactor tubes are externally cooled by a fluid, often water, medium passing there around, the horizontal temperature gradient naturally rises significantly within the first millimeter, usually 0.5 mm or less, from the reactor wall towards the reactor tube centre. After this ‘jump’ in temperature, the temperature difference gradient generally rises more gradually towards the centre of the tube. The temperature jump across the mantle depends on many factors, but it is generally a very small or thin reactor tube surface boundary layer compared with the full width of the reactor tube.

The process may also include fluid recycle in the reactor tubes. Catalyst-insert contact may be improved by the use of an inert gas or liquid flow there between, such as product recycle.

Without wishing to be restricted to a particular embodiment, the invention will now be described in further detail with reference to the drawings in which:

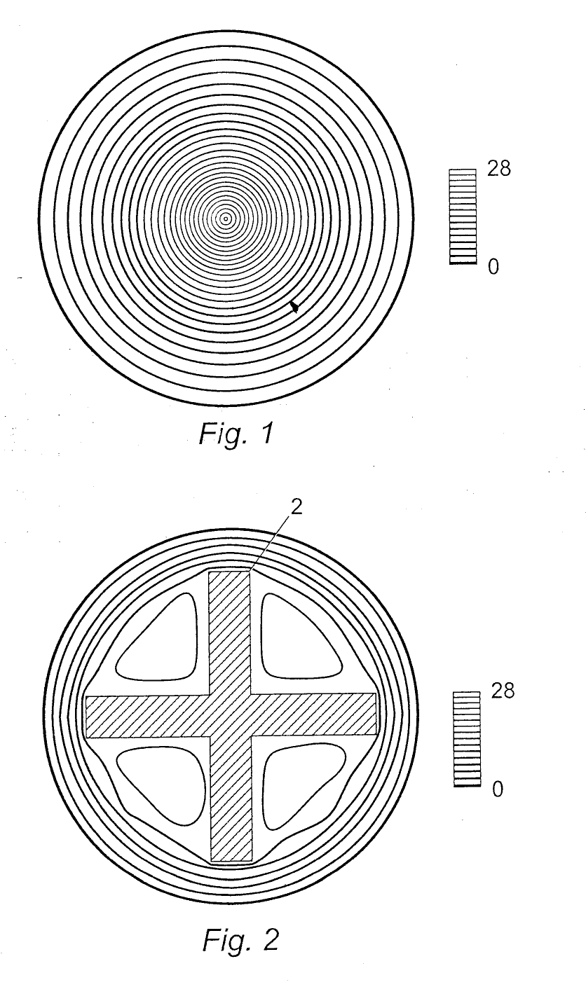

FIG. 1 is a schematic graphical representation of a radial temperature profile across a single tube usable in a multitubular reactor without an insert;

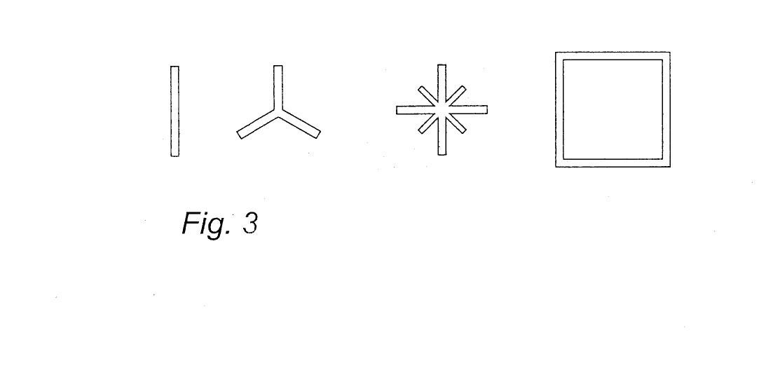

FIG. 2 is a schematic illustration of the same tube in FIG. 1, with an insert according to one embodiment of the present invention; and

FIG. 3 shows a cross-sectional view of a number of inserts useable in the present invention.

A typical catalytic multitubular reactor for carrying out catalytic processes such as those described herein comprises a normally substantially vertically extending vessel, a plurality of open-ended reactor tubes arranged in the vessel parallel to its central longitudinal axis of which the upper ends are fixed to an upper tube sheet or plate and in fluid communication with a fluid inlet chamber above the upper tube sheet and of which the lower ends are fixed to a lower tube plate and in fluid communication with an effluent collecting chamber below the lower tube sheet, optionally with a liquid supply means for supplying liquid to the fluid inlet chamber, gas supply means for supplying gas to the fluid inlet chamber, and an effluent outlet arranged in the effluent collecting chamber.

During normal operation the reactor tubes are filled with catalyst particles. To convert for example synthesis gas into hydrocarbons, synthesis gas is supplied through the fluid inlet chamber into the upper ends of the reactor tubes and passed through the reactor tubes. Effluents leaving the lower ends of the reactor tubes are collected in the effluent collecting chamber and removed from the effluent collecting chamber through the effluent outlet.

Such a multiple reactor can also be used for the catalytic conversion of a liquid in the presence of a gas.

A commercial multitubular reactor for such processes suitably will have a diameter of about 5 m, and between about 5000 reactor tubes with a diameter of about 60 mm, to about 15,000 reactor tubes (or even more) with a diameter of about 15 to 70 mm. The length of a reactor tube will substantially be about 5 to 15 m.

Typically, at least one of the reactants of an exothermic reaction is gaseous. Examples of exothermic reactions include hydrogenation reactions, hydroformylation, alkanol synthesis, the preparation of aromatic urethanes using carbon monoxide, Kölbel-Engelhardt synthesis, olefin oxidation (EO or PO), polyolefin synthesis, and Fischer-Tropsch synthesis. According to a preferred embodiment of the present invention, the exothermic reaction is a Fischer-Tropsch synthesis reaction.

The Fischer-Tropsch synthesis is well known to those skilled in the art and involves synthesis of hydrocarbons from a gaseous mixture of hydrogen and carbon monoxide, by contacting that mixture at reaction conditions with a Fischer-Tropsch catalyst.

Products of the Fischer-Tropsch synthesis may range from methane to heavy paraffinic waxes. Preferably, the production of methane is minimised and a substantial portion of the hydrocarbons produced have a carbon chain of at least 5 carbon atoms. Preferably, the amount of C₅₊ hydrocarbons is at least 60% by weight of the total product, more preferably, at least 70% by weight, even more preferably, at least 80% by weight, most preferably, at least 85% by weight.

Fischer-Tropsch catalysts are known in the art, and typically include a Group VIII metal component, preferably cobalt, iron and/or ruthenium, more preferably cobalt. Typically, the catalysts comprise a catalyst carrier. The catalyst carrier is preferably porous, such as a porous inorganic refractory oxide, more preferably alumina, silica, titania, zirconia or mixtures thereof.

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material.

The catalyst suitably has an average diameter of 0.5-15 mm. One form of catalyst is as an extrudate. Such extrudates suitably have a length of 2-10 mm, especially 5-6 mm, and a cross section suitably of 1-6 mm², especially 2-3 mm².

The catalytically active metal may be present in the catalyst together with one or more metal promoters or co-catalysts. The promoters may be present as metals or as the metal oxide, depending upon the particular promoter concerned. Suitable promoters include oxides of metals from Groups IIA, IIIB, IVB, VB, VIB and/or VIIB of the Periodic Table, oxides of the lanthanides and/or the actinides. Preferably, the catalyst comprises at least one of an element in Group IVB, VB and/or VIIB of the Periodic Table, in particular titanium, zirconium, manganese and/or vanadium. As an alternative or in addition to the metal oxide promoter, the catalyst may comprise a metal promoter selected from Groups VIIB and/or VIII of the Periodic Table. Preferred metal promoters include rhenium, platinum and palladium.

A most suitable catalyst comprises cobalt as the catalytically active metal and manganese and/or vanadium as a promoter.

The promoter, if present in the catalyst, is typically present in an amount of from 0.1 to 60 parts by weight per 100 parts by weight of carrier material. It will however be appreciated that the optimum amount of promoter may vary for the respective elements which act as promoter. If the catalyst comprises cobalt as the catalytically active metal and manganese and/or vanadium as promoter, the cobalt: (manganese+vanadium) atomic ratio is advantageously at least 12:1.

The Fischer-Tropsch synthesis is preferably carried out at a temperature in the range from 125° C. to 350° C., more preferably 175° C. to 275° C., most preferably 200° C. to 260° C. The pressure preferably ranges from 5 to 150 bar abs., more preferably from 5 to 80 bar abs.

Hydrogen and carbon monoxide (synthesis gas) is typically fed to a three-phase slurry reactor at a molar ratio in the range from 0.4 to 2.5.

Preferably, the hydrogen to carbon monoxide molar ratio is in the range from 1.0 to 2.5.

The gaseous hourly space velocity may vary within wide ranges and is typically in the range from 500 to 20,000 Nl/l/h, preferably in the range from 1000 to 10,000 Nl/l/h.

It will be understood that the skilled person is capable to select the most appropriate conditions for a specific reactor configuration and reaction regime. These include possible recycling of formed products such as gases and waxes.

Presently, the heat of exothermic reactions is only removed by a heat transfer fluid which is passed along the outer surfaces of the reactor tubes, (apart from the heat taken away by the temperature difference between the introduced reactants and withdrawn products.) This takes time, and so creates a raised temperature profile from the core to the mantle.

FIG. 1 shows simulation of a typical radial temperature profile within a single tube of a multitubular reactor carrying out a Fischer-Tropsch process, such as in a heavy paraffin synthesis. The temperatures are presented in degrees Celsius. The catalyst bed within the tube was a continuous conducting medium with conductivity of about 4 W/m/K, and the heat transfer coefficients to the mantle (the inner boundary of the reactor tube) were about 800 W/m/K.

As can be seen, FIG. 1 shows a typical temperature profile having a core temperature of the tube of about 16° C. higher than the temperature at the mantle.

FIG. 2 shows a graphic illustration of the same tube in FIG. 1, now including a heat transfer insert 2. The insert 2 has a geometric cross-section in the shape of a cross, having four equal fins radiating from a central axis. The insert could be formed from copper, iron, aluminium, steel or other suitable metals or materials, such as those used to form reactor tubes and walls.

As can be seen from FIG. 2, the core temperature of the tube is now about 6° C. above that at the mantle. A radial temperature difference of about 6° C. is a significant decrease in the difference compared with FIG. 1.

The insert 2 shown in FIG. 2 could be of other geometric shapes, including having a flat cross-section, or other star-like cross-sections having three, five, six etc. radial fins. Different fins may be of different lengths so as to create a multiple star cross-sectional shape effect. The insert could also be linear or twisted or helical, longitudinally. Some examples are shown in FIG. 3.

The thickness or other dimensions of a part or portion of the insert may be different of other parts, so as to vary the heat transfer ability and thus effect of the insert, either radially, longitudinally, or a combination of both. The insert provides the ability to transfer heat in the tube further than only from the centre to the mantle.

In particular, it is noted that the insert of the present invention may or may not be in physical contact with the tube wall.

The present invention provides a number of advantages. Firstly, it reduces temperature differentials, especially radial temperature differences as shown by FIG. 2. By transferring and conducting heat from the core to the mantle, the insert is significantly assisting the ability of the present invention to provide a more even radial temperature across the tube, than the general current method of cooling the process in the tube from the mantle inwardly, based on the outer cooling generally located around tubes in exothermic reactor tubes.

With suppression of the radial temperature difference, a more even and constant temperature across, possibly also along, all of the reactor tube provides a more even and constant activity of the catalyst therein, and so to a more even and constant efficiency of the process, and thus improvement in the process overall.

A second advantage is based on the constriction size effect. The size of the particles generally used to support the catalyst in exothermic reactions, and the effect of the pressure drop hereinbefore described, leads to packing considerations. Meanwhile it is known that smaller particles have better diffusion characteristics.

The insert of the present invention can provide the effect of dividing the tube to form a number of radial and/or axial defined tube portions, (such as the four longitudinal channels created by the insert 2 shown in FIG. 2). This division of the tube geometry creates smaller reactor tube volumes, which allows the use of smaller particles without adversely affecting pressure drop across the reactor. With smaller volumes, smaller particles can therefore be used without increasing the pressure drop.

One or more of the divided areas, possibly channels, may or may not include particles, or include the same particles as the other areas. This, along with possible differing insert geometry, can influence local catalytic activity.

A third advantage of the present invention is that the insert can be retrofitted to existing reactor tubes. Thus, the invention is immediately applicable to all multitubular reactors without expense or re-engineering. The invention thus provides a very cost-effective and flexible way to provide some suppression of radial temperature differences in comparison with any permanent internal structures in reactor tubes. Indeed, the insert does not need to be considered or incorporated in the design of new reactors and reactor tubes.

A further advantage is a decreased pressure drop. By inserting a straight insert (see FIG. 3, first drawing) or a cross-shaped insert it appears that the pressure decreases by about 10, respectively 25 percent.

The present invention also relates to a method to decrease the temperature profile of a catalyst bed in a reactor tube by using an insert as described above. The invention also relates to a method to reduce the pressure drop over a catalyst bed in a reactor tube by using an insert as described above.

As mentioned hereinbefore, the inserts do not need to extend throughout the length of the tube, or in every tube, such that their use, distribution and arrangement are flexible, allowing the user of the reactor tube to be very variable to suit different desired reactions and parameters. Some reactor tubes include integral structures, such as baffles, which may be reaction-specific, and difficult to adapt. Being integral, the baffles are not changeable.

Optionally, the insert may be perforated or otherwise include one or more apertures or cutaways to allow fluids such as gases and liquids in the tubes to circulate.

A further advantage of the present invention is the ability for a reactor using the present invention to have larger diameter reactor tubes. By being able to provide the same or possibly a reduced radial temperature difference across a tube diameter, a greater diameter tube can be used compared with the present reactor tubes.

A further advantage of the present invention is that the reduction or smearing out of the radial and/or axial temperature difference across the reactor tube provides a more constant temperature across and/or along the reactor tube, and thus increases efficiency, and increases the cell activity and selectivity of the catalyst bed as a whole.

The user can therefore better select and use the or each catalyst, to either optimise the reaction conditions, maximise the process yield, or both.

More generally, the present invention provides a means wherein heat, and possibly reactant flow, can be transferred within the reactor tube directionally, i.e. in any one or more than one direction desired, and at a rate as desired by working with the physical parameters of the insert, such as its nature, form, shape, etc. That is, it may be desired to transfer more heat longitudinally along a reaction path, e.g. along a reactor tube length, than radially.

The insert may be intermittent, or a number inserts used in one reactor tube, to effect differential radial and longitudinal heat transfer. The insert may also have parts of different thicknesses or materials, to assist this.

Most preferably, the insert of the present invention reduces temperature differentials in a reactor tube and reduces the pressure drop over a reactor, so as to minimise temperature differences and thus optimise reaction conditions and catalytic activity within the reactor tube, while minimising the pressure drop over the reactor tubes. Preferably all reactor tubes are provided with an insert, more preferably identical inserts.

The invention also relates to a Fischer-Tropsch process for the preparation of hydrocarbons from synthesis gas. In addition, the invention also relates to the hydrocarbons obtained in this process. Further, the invention also covers the hydrocarbons made in this way, as well as the hydrocarbons made from the Fischer-Tropsch hydrocarbons by means of hydrogenation, hydroisomerisation and/or hydrocracking of the original Fischer-Tropsch hydrocarbons. More especially the obtained products are claimed as kerosene, gasoil, waxy raffinate and/or base oils. Also (hydrogenated) solvents, detergent feedstock, drilling fluids are claimed.

1. A multitubular fixed bed reactor suitable for carrying out a catalytic process, which reactor includes a plurality of reactor tubes, one or more of which include a fixed bed of catalyst and is/are at least partially surrounded by a heat transfer medium, wherein the one or more reactor tubes each include at least one insert.

2. A reactor as claimed in claim 1 wherein the reactor tubes are elongate, and wherein the inserts are elongate.

3. A reactor as claimed in claim 1 wherein the inserts extend partly along the length of the reactor tubes.

4. A reactor as claimed in claim 1 wherein the inserts have a geometric cross-sectional shape, design or pattern.

5. A reactor as claimed in claim 1 wherein the inserts comprise radial fins extending at least partially perpendicularly to the direction of the reactor tube.

6. A reactor as claimed in claim 1 wherein the inserts have a central axis.

7. A reactor as claimed in claim 1 wherein the inserts are separate from each reactor tube.

8. A reactor as claimed in claim 1 wherein the inserts are formed from one or more of the group consisting of metals, and their alloys, metal-plated materials, and ceramic alloys.

9. (canceled)

10. A reactor as claimed in claim 1 wherein one or more inserts are adapted to divide the tube to form a number of defined tube portions.

11. A reactor tube for use in a multitubular reactor suitable for carrying our a catalytic process, wherein the reactor tube includes one or more inserts.

12. A process for the synthesis of hydrocarbons comprising the step of introducing synthesis gas into a multitubular reactor having a plurality of tubes in which the reactants are in contact with one or more catalysts, and one or more of the tubes is/are surrounded by at least one heat transfer medium, wherein at least one of the reactor tubes includes an insert.

13. A reactor as claimed in claim 1 wherein the inserts extend substantially along the length of the reactor tubes.

14. A reactor as claimed in claim 1 wherein the inserts extend beyond the length of the reactor tubes.

15. A reactor as claimed in claim 1 wherein the inserts are removable from the reactor tubes.

16. A reactor as claimed in claim 1 wherein the inserts include one or more apertures.

17. A reactor as claimed in claim 1 wherein the inserts are porous.

18. A reactor as claimed in claim 1 wherein the inserts comprise a ceramic, sintered or metal foam.
